(19) United States
(12) Patent Application Publication
(10) Pub. No.: US 2009/0139520 A1
(43) Pub. Date: Jun. 4, 2009

(54) APPARATUSES AND METHODS FOR PULMONARY DRUG DELIVERY

(75) Inventors: Phillip Weaver, Mouth of Wilson, VA (US); Lyndell Duvall, Fleetwood, NC (US); Jack Hebrank, Durham, NC (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: Next Safety, Inc., Jefferson, NC (US)

(21) Appl. No.: 11/950,154

(22) Filed: Dec. 4, 2007

(51) Int. Cl. A61M 16/00

(52) U.S. Cl. 128/203.12

(57) ABSTRACT

A pulmonary drug delivery device including a drug delivery tube that defines a flow path, a droplet ejection device configured to eject droplets of medication into the flow path, and a fan that generates airflow within the flow path, the airflow being configured to carry the ejected medication droplets along the flow path.

### BACKGROUND

The lung is the essential respiration organ in air-breathing vertebrates, including humans. Its principal function is to transport oxygen from the atmosphere into the bloodstream, and to excrete carbon dioxide from the bloodstream into the atmosphere. This exchange of gases is accomplished by a mosaic of specialized cells that form millions of tiny, thin-walled air sacs called alveoli. Beyond respiratory functions, the lungs also act as an efficient drug delivery mechanism. For example, the lungs have been used for centuries as a delivery mechanism for psychoactive drugs. One advantage of pulmonary drug delivery is that inhaled substances bypass the liver and the gastrointestinal tract and are therefore more readily absorbed into the bloodstream in comparison to orally-ingested medicines.

In recognition of the potential of pulmonary drug delivery, various efforts have been made toward developing effective pulmonary drug delivery devices. Current pulmonary drug delivery devices include metered dose inhalers (MDIs), dry powder inhalers (DPIs), and nebulizers. MDIs are pressurized hand-held devices that use propellants for delivering liquid medicines to the lungs. DPIs also use propellants, but deliver medicines in powder form. Nebulizers, also called “atomizers,” pump air or oxygen through a liquid medicine to create a vapor that is inhaled by the patient.

Each of the above-described devices suffer from disadvantages that decrease their attractiveness as a mechanism for pulmonary drug delivery. For example, when MDIs are used, medicine may be deposited at different levels of the pulmonary tree, and therefore may be absorbed to different degrees, depending on the timing of the delivery of the medicine in relation to the inhalation cycle. Accordingly, actual deposition of medicine in the lungs during patient use may differ from that measured in a controlled laboratory setting. Furthermore, a portion of the “metered dose” may be lost in the mouthpiece or the oropharynx.

Although DPIs reflect an effort to improve upon MDIs, small volume powder metering is not as precise as the metering of liquids. Therefore, the desired dosage of medicine may not actually be administered when a DPI is used. Furthermore, ambient environmental conditions, especially humidity, can adversely effect the likelihood of the medicine actually reaching the lungs.

Nebulizers may also exhibit unacceptable variability in delivered dosages, especially when they are of the inexpensive, imprecise variety that is common today. Although more expensive nebulizers are capable of delivering more precise dosages, the need for a compressed gas supply that significantly limits portability and the need for frequent cleaning to prevent bacterial colonization renders such nebulizers less desirable. Furthermore, the relatively high cost of such nebulizers also makes their use less attractive.

From the above, it can be appreciated that it would be desirable to have an improved pulmonary drug delivery system or device that avoids one or more of the above-described disadvantages.

Of the various applications in which such an improved pulmonary drug delivery system and device could be used, the delivery of nicotine as a method to achieve smoking cessation is one of the most compelling. The adverse health care consequences of smoking tobacco are enormous and incontrovertible. According the World Health Organization (WHO), tobacco is the second major cause of death in the world, currently accounting for one in ten deaths worldwide (5 million each year), and is the single largest preventable cause of disease and premature death. Of the 1.1 billion smokers in the world today, half will die from tobacco-related illness. For example, it is estimated that smoking will contribute to the death of one third of all Chinese males under 30 years old currently alive. In the United States, the 1999 National Health Interview Survey estimated that 46.5 million adults smoke and that 440,000 die each year from smoking related causes. In men, smoking is estimated to decrease life expectancy by 13.2 years and in women by 14.5 years.

Furthermore, it is now understood that cigarette smoke is not only harmful to the smoker, but also can affect the health of non-smokers when they passively inhale the smoke of other peoples' cigarettes. Such “secondhand smoke” is a risk factor for numerous types of adult ailments including lung cancer, breast cancer, and heart disease. Secondhand smoke exposure also increases the risk of various diseases in children and infants.

Despite recognizing the health risks associated with their habit, smokers continue to smoke. The primary reason for this phenomenon relates to the effect that nicotine has on the central nervous system (CNS). At low serum levels, nicotine provides stimulatory effects, primarily through activation of the locus ceruleus within the cerebral cortex. Such stimulatory effects include increased concentration, decreased anxiety, improved mood, decreased appetite, and improved memory. At high serum levels, nicotine activates the limbic system and produces a sense of euphoria, commonly referred to as a “buzz.” Cigarette smokers are accustomed to achieving both of these effects.

After inhaling cigarette smoke, nicotine is absorbed across the alveolar membrane in the lungs, leading to a rapid rise of serum nicotine levels within a few seconds. Within five minutes of smoking, the average maximum concentration of nicotine in arterial blood rises to 49 nanograms per milliliter (ng/ml), thereby providing the euphoric buzz. As nicotine levels fall, the stimulant effects predominate for the next 1-2 hours. Soon after, however, withdrawal symptoms begin to develop. These symptoms include irritability, anger, impatience, restlessness, difficulty concentrating, increased appetite, anxiety, and depressed mood. Such withdrawal symptoms are normally relieved by smoking the next cigarette, thereby creating a potentially endless cycle.

Over the years, many efforts have been made to develop effective means for assisting smokers in quitting. Currently, there are several Federal Drug Administration (FDA) approved nicotine replacement treatments (NRTs) intended for use in smoking cessation available both over-the-counter and as a prescription. Significantly, none of those NRTs deliver significant amounts of nicotine to the alveolar level of the lungs. Instead, they rely on the absorption of nicotine across the skin or across the nasal, buccal, or oropharyngeal mucosa. As a result, absorption is much slower and much less efficient than that typical of smoking and therefore leads to slower and much lower peak nicotine concentrations compared to that produced by cigarettes. Notably, this is true for existing nicotine inhalers, which are purported to have delivery characteristics most like cigarettes. Studies have confirmed that nicotine absorption resulting from use of such inhalers primarily occurs across the buccal mucosa, not the lungs, and that the arterial nicotine concentration spike that results from cigarette smoking does not occur with such inhalers.

The peak serum levels achieved with the current NRTs may be adequate to ameliorate or prevent withdrawal symptoms. However, they do little to satisfy the acute craving for the “buzz” created by the rapid onset and high peak serum nicotine levels typical of tobacco smoke. This may be the primary reason why so few habitual smokers that have used NRT have achieved long-term success. Instead, such persons typically give in to the persistent cravings, which currently can only be satisfied through smoking.

Given the enormity of the health problems caused by smoking, it is agreed upon by physicians and laypersons alike that the best thing that smokers can do is quit smoking. However, given the limited success that previous cessation solutions have had, it is clear that more effective alternatives are needed. It stands to reason that an alternative capable of providing nicotine to the user in ways analogous to smoking could save numerous lives.

### BRIEF DESCRIPTION OF THE DRAWINGS

The disclosed apparatuses and methods can be better understood with reference to the following drawings. The components in the drawings are not necessarily to scale.

FIG. 1 is front perspective view of an embodiment of a device for pulmonary drug delivery.

FIG. 2 is a rear perspective view of the device of FIG. 1.

FIG. 3 is a front perspective view of the device of FIG. 1 with a front cover of the device removed.

FIG. 4 is an exploded perspective view of the device of FIG. 1.

FIG. 5 is side perspective view of a drug delivery member of the device of FIG. 1.

FIG. 6 is a cross-sectional side view of the drug delivery member of FIG. 5.

FIG. 7 is a side perspective view of a medicine storage and delivery unit of the drug delivery member of FIG. 5.

FIG. 8 is a first rear perspective view of the drug delivery member of FIG. 5, shown with an electrical conductor decoupled from the member.

FIG. 9 is a second rear perspective view of the drug delivery member of FIG. 5, shown with the electrical conductor coupled to the member.

FIG. 10 is bottom perspective view of the medicine storage and delivery unit shown of FIG. 7, illustrating a droplet ejection device of the unit.

FIG. 11 is a top view of an alternative embodiment of a drug delivery member that can be used in the device of FIG. 1.

FIG. 12 is an end view of the drug delivery member of FIG. 11.

### DETAILED DESCRIPTION

As described above, it would be desirable have a pulmonary drug delivery system or device that is effective in enabling absorption of medicines, such as nicotine, via the lungs. Embodiments of a pulmonary drug delivery device are described in the following disclosure.

Disclosed herein are various embodiments of apparatuses and methods for pulmonary drug delivery. It is noted that those embodiments comprise mere implementations of the disclosed inventions and that alternative embodiments are both possible and intended to fall within the scope of the present disclosure.

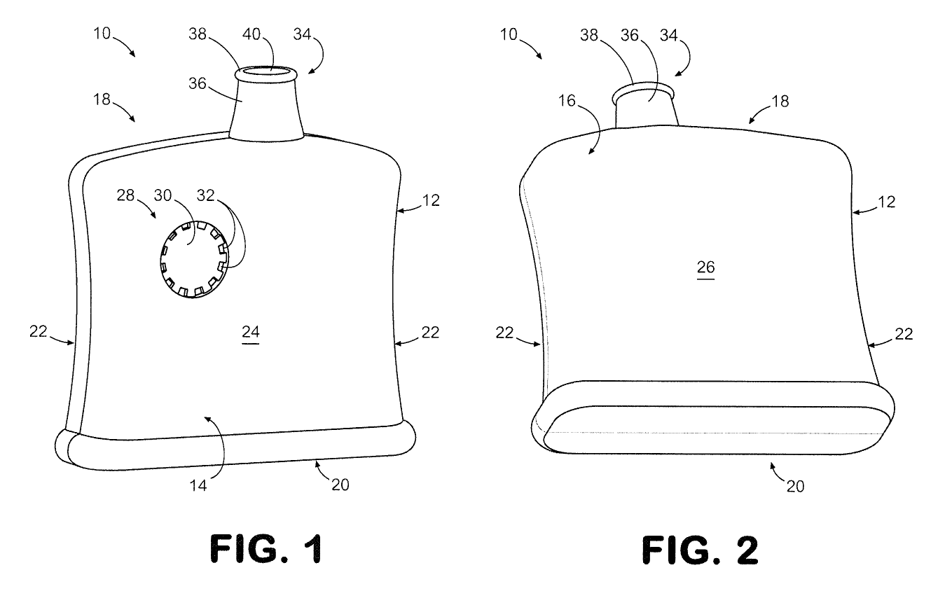

Referring to the drawings, in which like numerals indicate corresponding parts throughout the several views, FIGS. 1 and 2 illustrate an example pulmonary drug delivery device 10. In at least some embodiments, the device 10 comprises a portable (e.g., handheld) device that can be easily carried with the user throughout the day so as to be available whenever needed. The device 10 includes an outer housing 12 that generally comprises a front side 14, a rear side 16, a top side 18, a bottom side 20, and opposed lateral sides 22. In some embodiments, the device 10 comprises a front cover 24 that defines the front side 14 and portions of the top side 18, bottom side 20, and opposed lateral sides 22, and a rear cover 26 that defines the rear side 16 and portions of the top side 18, bottom side 20, and opposed lateral sides 22. Provided on the front side 14, for example formed within the front cover 24, is an air inlet 28 that enables ambient air to flow from the environment in which the device 10 is used into an interior space of the device. In the illustrated embodiment, the inlet 28 comprises a generally circular depression 30 that includes a plurality of openings or slots 32 that are formed around the periphery of the depression.

Extending from the top side 18 of the housing 12 is a mouthpiece 34 that is used to deliver medicine to a patient who uses the device 10 (i.e., a “user”). In the embodiment of FIGS. 1 and 2, the mouthpiece 34 comprises a hollow, frustoconical member 36 that terminates in a rounded lip 38 that the user can place in his or her mouth. As is apparent from FIG. 1, the mouthpiece 34 includes an opening 40 at its end that serves as an outlet for the device 10.

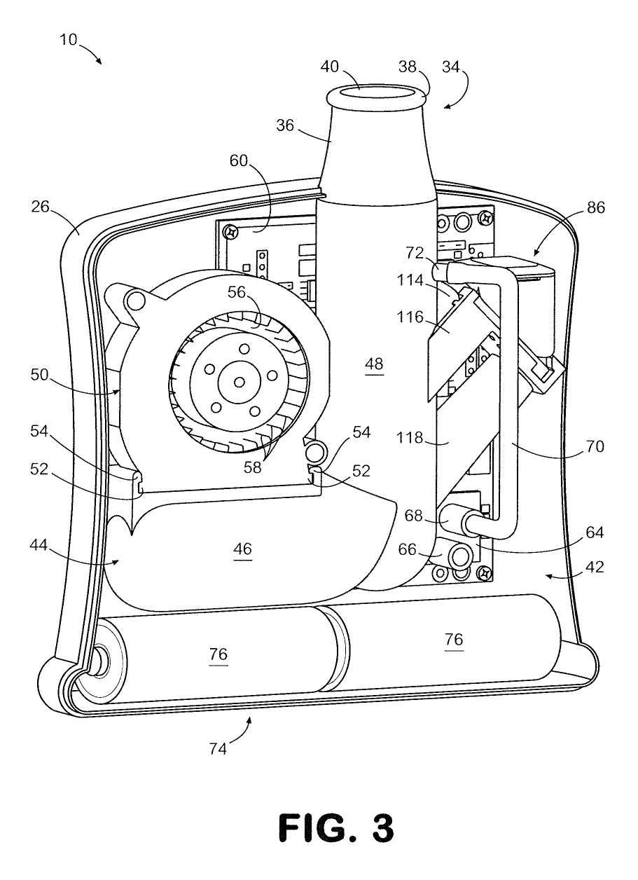

FIG. 3 is a front perspective view of the device 10 with the front cover 24 removed to reveal the interior space 42 defined by the device and, more particularly, by its two covers 24, 26. As indicated in FIG. 3, provided within the interior space 42 is a drug delivery member 44. As is also indicated in FIG. 3, the drug delivery member 44 is generally L-shaped and therefore comprises a first or lower tube 46 that is in fluid communication with a second or upper tube 48. In the illustrated embodiment, the lower tube 46 is horizontally arranged and the upper tube 48 is vertically arranged such that the bottom and upper tubes form a sharp angle between them. As described in greater detail below, that sharp angle both facilitates suspension of ejected droplets of medicine within air that flows through the drug delivery member 44 and desired evaporation of the droplets before they exit the drug delivery member. In some embodiments, the bottom and upper tubes 46, 48 are unitarily formed together, for example using an injection molding process. Likewise, the mouthpiece 34 can be unitarily formed with the upper tube 48 such that each of the lower tube 46, upper tube 48, and mouthpiece 34 are formed from a single, continuous piece of material, such as a plastic material. Given that they form a continuous tube in such an arrangement, the lower tube 46, upper tube 48, and mouthpiece 34 can be generally considered to form a drug delivery tube.

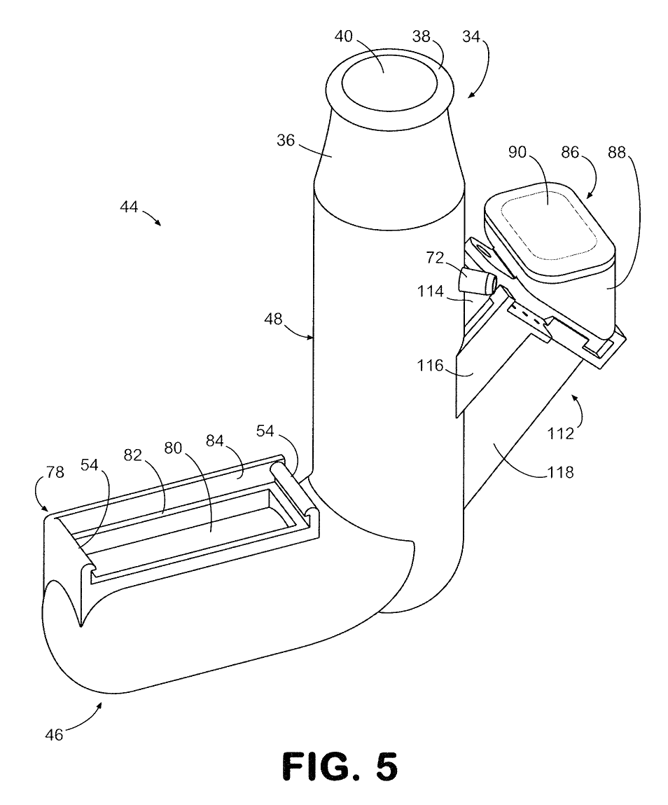

Also provided within the interior space 42 is a fan 50 that is used to generate airflow within the drug delivery member 44. As indicated in FIG. 3, the fan 50 can, in some embodiments, mount to the drug delivery member 44, for example to the lower tube 46. In such embodiments, the fan 50 can comprise mounting flanges or lugs 52 that are retained by brackets 54 provided on the lower tube 46 (see FIG. 5). Irrespective of how or whether the fan 50 is mounted to the drug delivery member 44, the fan comprises an inlet 56 through which air is drawn into the fan and an outlet (not shown) from which the drawn air is exhausted from the fan at an increased velocity into the drug delivery member. In some embodiments, the fan 50 comprises a centrifugal blower that includes an internal impeller having blades 58 that force air out from the fan in direction perpendicular to the inlet 56. By way of example, the fan 50 comprises a pulse-width-modulated (PWM) centrifugal blower that outputs approximately 40 to 160 standard liters per minute (slm).

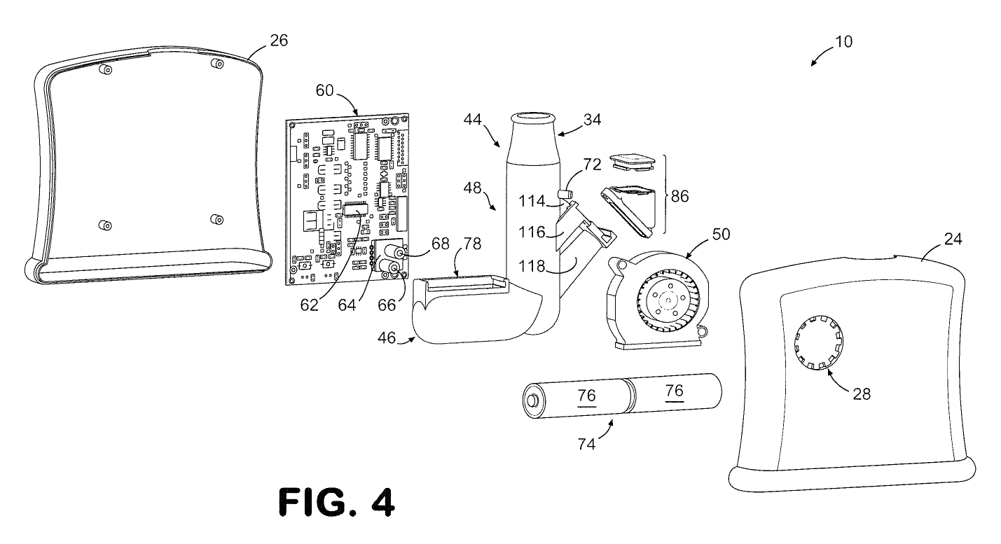

Further provided within the interior space 42 is a circuit board 60, which is more clearly shown in the exploded view of FIG. 4. The circuit board 60 generally comprises the logic that controls operation of the pulmonary drug delivery device 10. That logic can, for example, comprise a controller 62, such as a microcontroller or other processing means, that controls the operation of various components of the device 10, including the fan 50 and a droplet ejection device used to inject medicine into the airflow created by the fan. Also provided on the circuit board 60 is a pressure sensor 64, such as an integrated circuit (IC) differential pressure sensor. The pressure sensor 64 is connected to two ports: an atmospheric port 66 that is in fluid communication with the environment via the inlet 28, and an airflow port 68 that can be placed in fluid communication with the interior of the drug delivery member 44 using a coupling tube, such as tube 70 identified in FIG. 3. As indicated in FIG. 3, the tube 70 can extend from the port 68 to a further port 72 provided on (e.g., unitarily formed with) the upper tube 48 of the drug delivery member 44. With such an arrangement, the pressure sensor 64 can detect pressure drops within the upper tube 48 that are indicative of a user drawing in air from the mouthpiece 34 (i.e., inhaling from the mouthpiece).

With continued reference to FIGS. 3 and 4, the interior space 42 further includes an internal power supply 74. In some embodiments, the power supply 74 comprises alkaline batteries 76. By way of example, the batteries 76 comprise two “AA” type batteries. Irrespective of what particular type of power supply is used, the power supply 74 provides power to the fan 50, the circuit board 60, and the above-mentioned droplet ejection device. Such power can be provided with various electrical conductors (not shown) that extend from the power supply 74.

FIG. 5 illustrates the drug delivery member 44 in greater detail. As described above, the drug delivery member 44 includes a first or lower tube 46 in fluid communication with a second or upper tube 48, which is in fluid communication with a mouthpiece 34 that forms an opening 40 that functions as an outlet of the drug delivery member 44. As indicated in FIG. 5, the lower tube 46 comprises a fan mount 78 that includes a further opening 80 that functions as an inlet of the drug delivery member 44. As mentioned above, the fan mount 78 includes brackets 54 that are adapted to retain flanges or lugs 52 of the fan 50. In addition, the fan mount 78 comprises a support surface 82 that mates with and supports the fan 50 adjacent the fan's outlet, and a rear wall 84 that limits the depth of insertion of the fan relative to the fan mount 78. With such an arrangement, the fan 50 can be placed in the operating position shown in FIG. 3 by first positioning the fan lugs 52 inside of (e.g., below) the brackets 54 of the fan mount 78 and then sliding the fan along the support surface 82 until the fan abuts the rear wall 84. Once the fan 50 is placed in that position, the fan's outlet is directly adjacent the opening 80 such that air exhausted by the fan is blown directly into the lower tube 46. As described below, the air is blown in a direction that, at least in some embodiments, forms an acute angle with a longitudinal axis of the lower tube 46.

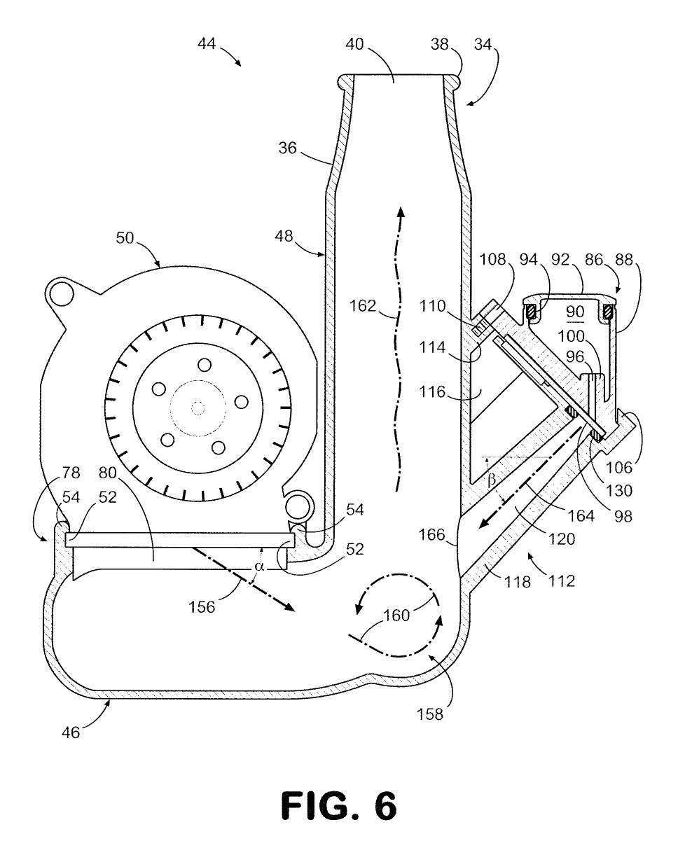

As is further indicated in FIG. 5, the drug delivery member 44 also comprises a medicine storage and delivery unit 86 that, in the illustrated embodiment, is supported by a support structure that extends from the upper tube 48. The medicine storage and delivery unit 86 comprises a medicine container 88 that, as indicated in the cross-sectional view of FIG. 6, defines an interior space 90 that can be partially or wholly filled with medicine intended for delivery to the respiratory system of the device user. A removable cap 92 is provided on the container 88 to reduce or prevent leakage and/or evaporation of the medicine provided within the container. In some embodiments, the cap 92 comprises a sealing member 94, such as an O-ring, that forms an airtight seal between the cap and the container 88. With further reference to FIG. 6, a passage 96 extends from the interior space 90 of the container 88 to a droplet ejection device 98, that is integrated into the medicine storage and delivery unit 86. As shown in FIG. 6, the passage 96 can be formed in a boss 100 that extends upwardly into the interior space 90.

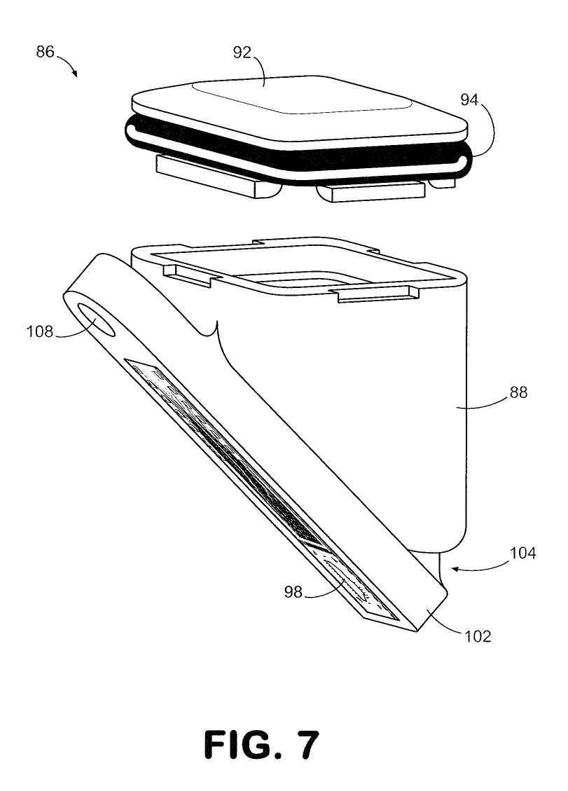

Turning to FIG. 7, the medicine storage and delivery unit 86 is provided with a support member 102 that facilitates mounting of the unit to the above-mentioned support structure. A lip 104 is formed by the support member 102 adjacent its bottom end that is adapted to be secured by a retainer clip 106 that is formed on the support structure (FIG. 6). Adjacent the top end of the support member 102 is an opening 108 that is adapted to receive a fastening element, such as a screw, that can be passed through the support member and threaded into a further opening 110 provided in the support structure (FIG. 6). Accordingly, the medicine storage and delivery unit 86 can be mounted to the support structure by first positioning the bottom lip 104 underneath the retainer clip 106 and then securing the top end of the unit to the support structure using the fastening element (not shown).

The above-mentioned support structure will now be described with reference to FIGS. 5, 6, and 8. The support structure is generally identified in FIGS. 5 and 6 by reference numeral 112. In the illustrated embodiment, the support structure 112 comprises a boss 114, two laterally opposed struts 116, and a medicine injection tube 118, each of which extends at an upward diagonal angle from the upper tube 48. The boss 114 defines the opening 110 that receives the fastening element used to secure the medicine storage and delivery unit 86 to the support structure 112. The struts 116 act as posts that provide lateral support and stability for the medicine storage and delivery unit 86. The medicine injection tube 118 also provides support and stability for the medicine storage and delivery unit 86, and further defines a pathway 120 (FIG. 6) for droplets ejected from the droplet ejection device 98 to travel before reaching a flow path defined by the bottom and upper tubes 46, 48.

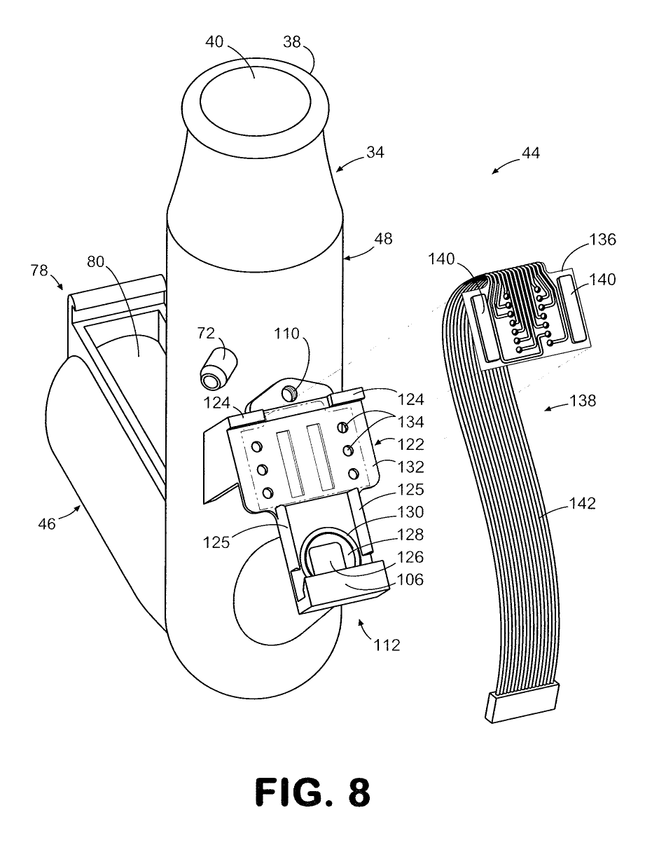

Referring next to FIG. 8, the support structure 112 defines a platform 122 on which the medicine storage and delivery unit 86 is supported. The platform 122 is generally planar and lies within a plane that forms an acute angle with the longitudinal axis of the upper tube 48. Extending upwardly from the platform 122 are upper and lower alignment tabs 124 and 125, respectively, that act as lateral boundary walls for the medicine storage and delivery unit 86. Located near the bottom end of the platform 122 between the lower alignment tabs 125 is an opening 126 that leads to the pathway 120 formed in the medicine injection tube 118 (FIG. 6). In the illustrated embodiment, the opening 126 is generally rectangular (e.g., square) with rounded corners. In some embodiments, the pathway 120 likewise has a generally rectangular (e.g., square) square cross-section with rounded corners. As indicated in FIG. 6, the pathway 120 can be tapered such that it widens as the pathway is traversed from the droplet ejection device 98 to the flow path defined by the drug delivery tube. With further reference to FIG. 8, surrounding the opening 126 is a generally circular recess 128 in which a further sealing member 130, such as an O-ring, is positioned. When provided, the sealing member 130 forms an airtight seal between the droplet ejection device 98 and the pathway 120 and prevents leakage of medicine onto electrical contacts of the droplet ejection device.

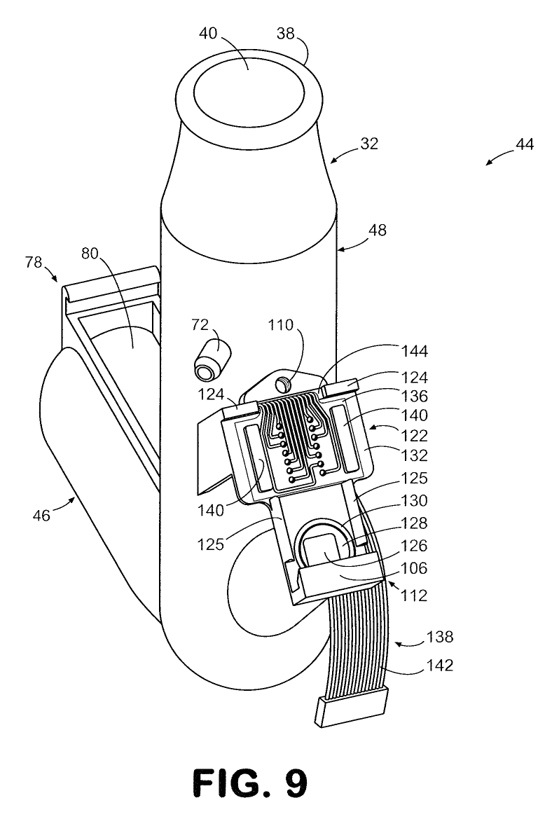

Adjacent the top end of the platform 122 is a seat 132 that is adapted to receive and support a head 136 of an electrical cable 138 that electrically couples the droplet ejection device 98 (FIG. 6) to the circuit board 60 (FIG. 3 and 4). In the illustrated embodiment, the seat 132 comprises various mounting holes 134 that facilitate mounting of the electrical cable head 136 to the platform 122. Exemplary seating of the cable 138 is illustrated in FIG. 9. As indicated in that figure, the head 136 of the cable 138 is positioned on the seat 132 and is secured thereto with attachment elements 140 that extend through the head and into the mounting holes 134. As is further indicated in FIG. 9, the ribbon 142 of the cable 138 extends from the head 136 and through a slot 144 formed in the platform 122 adjacent the opening 110 such that control signals, for example encompassed in a waveform, can be sent from the circuit board 60 to the droplet ejection device 98.

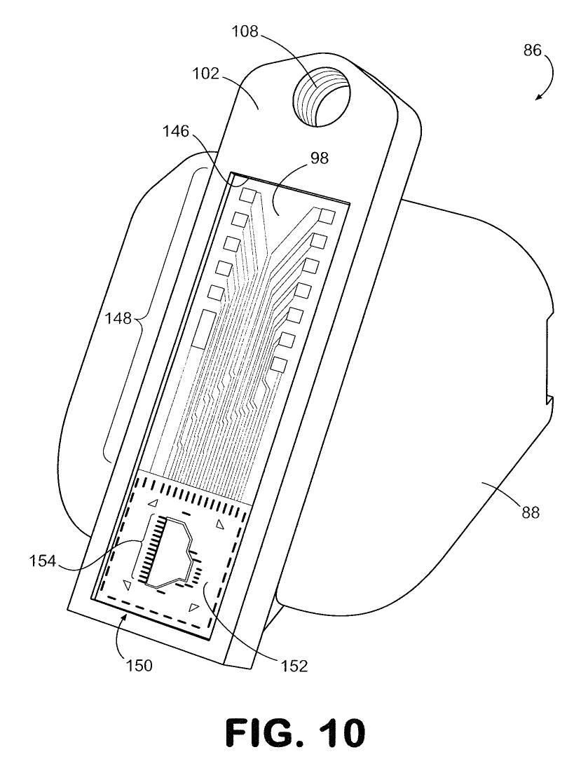

FIG. 10 shows the underside of the medicine storage and delivery unit 86 and the droplet ejection device 98 thereof. As indicated in FIG. 10, the droplet ejection device 98 comprises a rectangular circuit board that is positioned within a rectangular recess 146 formed in the underside of the support member 102. Formed within the droplet ejection device 98 are a plurality of traces 148 that electrically couple an ejection head 150 of the device with contacts provided on the electrical cable head 136. The ejection head 150 comprises a nozzle plate 152 that defines a plurality of nozzles 154 from which droplets of medicine can be ejected. By way of example, the ejection head 150 comprises approximately 5 to 100 such nozzles. Associated with each nozzle 154 is an ejection element (not shown), such as a heater resistor, that, when activated, causes ejection of one of more droplets of medicine. When heater resistors are used, thin layers of medicine within firing chambers (not shown) formed within the ejection head 150 are superheated, causing explosive vaporization and ejection of droplets of medicine through the nozzles 154. Ejection of the droplets creates a capillary action that draws further medicine within the firing chambers such that the droplet ejection device can be repeatedly fired. The size of the droplets depends upon the particular configuration of the ejection head 150. In some embodiments, primary droplets ejected from the nozzles 154 are approximately 0.1 to 300 picoliters (pi) in volume, or approximately 0.1 to 10 microns (μm) in diameter. Such volumes and sizes can be reproduced with great precision and accuracy with the ejection head 150. Indeed, in regard to precision, testing has confirmed that approximately half of the droplets that are ejected are within approximately 500 nanometers of each other in terms of diameter. The droplets are typically ejected from the nozzles at a velocity of approximately 1 to 7 meters per second (mls).

Example configurations for the pulmonary drug delivery device 10 having been described in the foregoing, examples of operation of the device will now be described. As explained above, the device 10 can be activated to deliver medicine to the respiratory system of the user upon detecting user inhalation as indicated by a drop in pressure within the upper tube 48 of the drug delivery member 44. The pressure drop can be detected by the pressure sensor 64 and an appropriate detection signal can then be sent from the sensor to the device microcontroller 62. The microcontroller 62 can then activate the fan 50 to cause it to draw in air from the environment, for example through the inlet 28 provided in the front cover 24, and exhaust the air through the opening 80 of the lower tube 46, as indicated by flow arrow 156 in FIG. 6. By way of example, the air is exhausted at a velocity of approximately 0.5 to 3 m/s.

Due to the nature of the fan 50, the air is exhausted at a relatively precise angle relative to the lower tube 46. By way of example, the exhaust angle, a, is approximately 10 to 40 degrees relative to a horizontal direction that is parallel to the longitudinal axis of the lower tube 46. As mentioned above, a sharp angle is formed between the lower tube 46 and the upper tube 48. By way of example, that angle is approximately 70 to 120 degrees, for example approximately 90 degrees. Due to that sharp angle, the air exhausted by the fan 50 impinges upon the walls of the upper tube 48 and becomes highly turbulent within a turbulence zone 158 adjacent the intersection between the lower and upper tubes 46, 48 (i.e., at the sharp “bend” of the drug delivery tube). As is schematically indicated by flow arrows 160, the air vigorously circulates with the turbulence zone 158 before being forced up through the upper tube 48, as indicated by flow arrow 162.

Simultaneous to or soon after activation of the fan 50, the microcontroller 62 activates the droplet ejection device 98 to cause droplets of medicine to be ejected from the nozzles 154 of the ejection head 150. In some embodiments, the nozzles 154 are selectively activated to ensure a desired separation in terms of both distance and time. For example, the nozzles 154 can be activated such that only non-adjacent nozzles eject in sequence and a period of at least approximately 150 to 500 microseconds (ps) passes between firing of any two nozzles. Such an activation scheme ensures that the droplets are physically spaced to a degree at which evaporation of a droplet is not significantly influenced by the proximity of one or more other droplets.

Irrespective of the nozzle activation scheme that is implemented, the ejected droplets travel along the pathway 120 of the medicine injection tube 118 in the direction of arrow 164, which forms an angle, β, of approximately 30 to 60 degrees relative to the horizontal direction and which is generally opposite to the direction of the airflow generated by the fan 50. As indicated in FIG. 6, the droplets are injected directly into the turbulence region 158 so that the droplets enter the airflow within the drug delivery tube at the point of highest turbulence. Injection of the droplets at that site facilitates controlled evaporation that, in turn, shrinks the droplets so that, by the time the droplets reach the opening 40 of the mouthpiece 32, the droplets at or near optimal size for absorption by the lungs.

In order to achieve effective systemic absorption, it is normally desirable to deliver a medicine directly to the alveoli located deep within the lung structure where transport to the bloodstream is most quickly and efficiently accomplished. Lung deposition curves, such as those published by the International Commission on Radiological Protection (ICRP), indicate that the locations within the pulmonary tree in which inhaled particles are deposited depends to a substantial degree upon particle size. Specifically, lung deposition curves based on both theoretical modeling and experimental data typically show that particle deposition rates in the alveolar regions of the lung are greatest for particles having a diameter of approximately 1 to 3 μm. In view of this, the device 10 can be configured to deliver droplets having a diameter of approximately 1 to 3 μm from the opening 40 of the mouthpiece 34. In other embodiments, the droplets have even smaller exit diameters, for example approximately 0.1 to 1 μm, to enable hygroscopic growth of the droplets within the respiratory tract.

With further reference to FIG. 6, it is noted that the droplets exit the droplet injection tube 118 at a location and angle at which the droplets must travel a relatively long distance before reaching the walls of the lower tube 46. In addition to increasing flight time, which further assists in the evaporation process, such an arrangement reduces the likelihood that the droplets will be deposited upon the tube walls instead of being delivered to the user's respiratory tract.

After the desired quantity of medicine has been injected into the airflow during the current inhalation cycle, ejection of medicine droplets ceases and the fan 50 is powered down. The process can then be repeated for further inhalation cycles of the user until a desired dosage of medicine has been administered. If desired, the entire process can be repeated at a later time, such as later that day or the next day. In some embodiments, appropriate controls can be integrated into the device 10 to limit the frequency with which the medicine can be administered. For example, the microcontroller 62 can be programmed to limit operation of the device 10 once every hour, once every few hours, once each day, and the like.

As mentioned above, it may be desirable to deliver droplets having a diameter of approximately 1 to 3 μm from the opening 40 of the mouthpiece 34. Notably, the size of the droplets that are ejected from the droplet ejection device 98 may be outside of that range. For example, environmental conditions, such as temperature, humidity, and pressure, can cause the ejected droplets to shrink or grow. In some embodiments, measures may be taken, substantially in real time, to control the size of the droplets relative to feedback that is collected by the device 10. Such feedback can comprise, for example, one or more of the current atmospheric temperature, humidity, and pressure, or the size of the droplets that are being delivered. In the former case, the device 10 comprises an open feedback loop and, in the latter case, the device comprises a closed feedback loop. Irrespective of which feedback scheme is used, the actions to be taken can be determined through reference to a look-up table or through application of an appropriate algorithm, either of which can be stored within memory provided on the circuit board 60. In cases in which the current atmospheric temperature, humidity, and pressure are to be measured (i.e., open-loop feedback), the circuit board 60 can also include appropriate sensors for detecting those conditions.

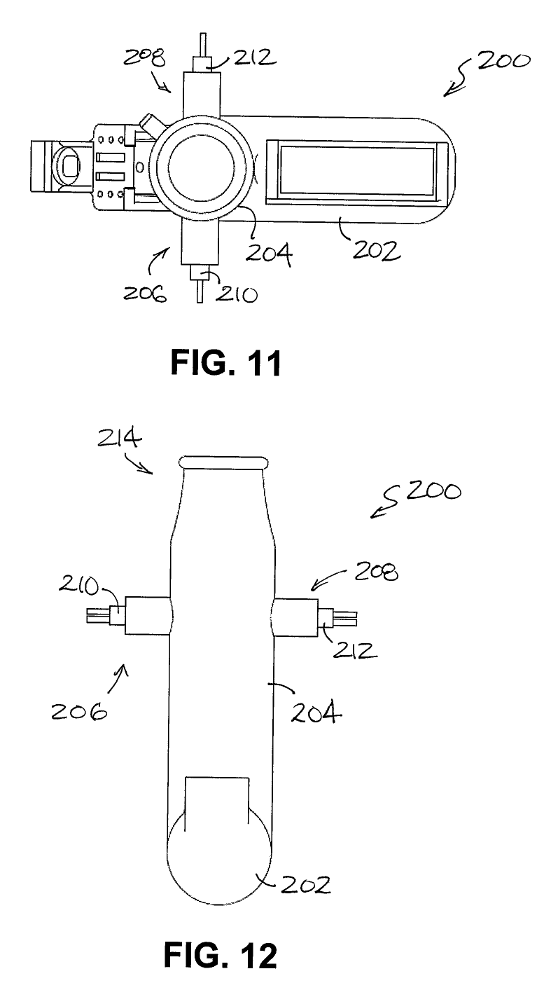

In cases in which the size of the droplets are to be measured (i.e., closed-loop feedback), the device 10 can comprise appropriate droplet size sensing apparatus. FIGS. 11 and 12 illustrate an alternative drug delivery member 200 that includes such apparatus. As indicated in those figures, the drug delivery member 200 is similar in many ways to the drug delivery member 44. Therefore, the drug delivery member 200 comprises a first or lower tube 202 in communication with a second or upper tube 204. Provided on the upper tube 204, however, are two ports 206 and 208 that provide access to the interior of the upper tube. Associated with the first port 206 is a light source 210 and associated with the second port 208 is a light detector 212. By way of example, the light source 210 comprises a light-emitting diode (LED) that emits laser light toward the light detector 212, which can comprise a photoelectric sensor.

The light source 210 and light detector 212 together comprise a droplet size sensing apparatus configured to capture light data regarding the droplets flowing through the upper tube 204. As indicated in FIG. 12, both the light source 210 and the light detector 212 can be located at a position near the end of the upper tube 204 adjacent the mouthpiece 214 so as to provide data relevant to the size of the droplets just before they exit the mouthpiece and enter the patient. Accordingly, the approximate size of the droplets being administered can be determined and adjustments can be made to modify the sizes of later-ejected droplets, if necessary.

Generally speaking, the size of the droplets can be controlled during droplet formation, after droplet formation, or both. During droplet formation, certain parameters can be controlled to alter the size of the droplets that are ejected. In some cases, the droplet size may not necessarily be the same as the size of the nozzle orifice. For example, droplets that are smaller or larger than the nozzle orifice may be produced. After droplet formation, certain other parameters can be controlled to change the size of the generated droplets. For example, the droplets can be reduced in size downstream of the nozzle orifice through controlled evaporation. Using such processes, a droplet ejection device having relatively large (e.g., approximately 10 to 30 μm) orifices can still be used to deliver substantially smaller (e.g., approximately 1 to 3 μm) droplets.

Regarding droplet formation, it has been determined that relatively small droplets can be generated when the liquid from which the droplets are formed is maintained at an elevated temperature. Such elevated temperatures decrease both the viscosity and surface tension of the liquid, which translates into smaller droplets being ejected. Notably, the composition of the liquid (e.g., medication solution) can also affect droplet size. Therefore, results may vary depending upon the nature of the medication being administered.

As mentioned above, droplet size can be controlled after formation. The exercise of such control may generally be referred to as post-processing of the droplets. Such post processing can include controlling the rate at which the ejected droplets evaporate during their flight to the user's respiratory tract. As indicated above, factors or parameters that have an impact droplet evaporation include air temperature, humidity, and pressure. Therefore, the evaporation rate can be controlled through manipulation of one or more those parameters. For example, droplet size can be reduced by heating the air that flows through the system. As a further example, the size of the droplets can be reduced or increased by respectively decreasing or increasing the humidity of the air that delivers the droplets.

Appropriate apparatuses to control parameters such as liquid temperature, air temperature, and air humidity can be added to the delivery device 200, as desired.

Various modifications can be made to the embodiments described in the foregoing. For example, in one alternative embodiment, an extension tube can be connected to the mouthpiece of the device and used to increase the distance between the device housing and the point at which medicine enters the user's mouth. In another alternative embodiment, the cap to the medicine container can include a vent port that equalizes the pressure within the container with that of the surrounding environment. In a further alternative embodiment, a screen can be placed over the passage formed within the container to filter particulate matter that could clog the droplet ejection device and/or to reduce surface tension that could interfere with the flow of medicine to the drug ejection device.

It is further noted that appropriate regulatory measures can be taken to avoid abuse of the device or the medicine(s) that the device is intended to administer. For example, each medicine storage and delivery unit can be sold separately as one-time use component that comprises identification data that can be read by the pulmonary drug delivery device when the unit is installed on the drug delivery member. If the device microcontroller determines from the identification data that the medicine storage and delivery unit does not contain a medicine for which the device has been prescribed, for example by a doctor, operation of the device can be disabled.

Finally, it is noted that absolute spatial terms such as “horizontal” and “vertical” have been used herein relative to the orientations of the device components shown in the drawings. Therefore, it is to be understood that such terms may not strictly apply in cases in which the orientation of the device is changed from that shown in the figures.

1. A pulmonary drug delivery device, comprising:
a drug delivery tube that defines a flow path;
a droplet ejection device configured to eject droplets of medication into the flow path; and
a fan that generates airflow within the flow path, the airflow being configured to carry the ejected medication droplets along the flow path.

2. The pulmonary drug delivery device of claim 1, wherein the drug delivery tube comprises a first tube in fluid communication with a second tube and wherein the first and second tubes form a sharp angle between each other.

3. The pulmonary drug delivery device of claim 2, wherein the first and second tubes form an angle between each other of approximately 30 to 60 degrees.

4. The pulmonary drug delivery device of claim 2, wherein the first and second tubes form an angle between each other of approximately 90 degrees.

5. The pulmonary drug delivery device of claim 2, wherein a zone of relatively high turbulence exists adjacent an intersection of the first and second tubes due to the sharp angle formed between the first and second tubes.

6. The pulmonary drug delivery device of claim 1, wherein the droplet ejection device comprises an ejection head including a plurality of nozzles and a plurality of ejection elements that cause droplets to be selectively ejected from the nozzles.

7. The pulmonary drug delivery device of claim 6, wherein the ejection elements comprise heater resistors.

8. The pulmonary drug delivery device of claim 1, wherein the fan comprises a centrifugal blower.

9. The pulmonary drug delivery device of claim 1, further comprising a pressure sensor configured to sense a pressure drop within the drug delivery tube.

10. The pulmonary drug delivery device of claim 9, further comprising a microcontroller that activates the fan and the droplet ejection device in response to the pressure drop sensed by the pressure sensor.

11. The pulmonary drug delivery device of claim 1, further comprising an internal power supply that powers the droplet ejection device and the fan.

12. The pulmonary drug delivery device of claim 1, further comprising a medicine container configured to supply medicine to the droplet ejection device.

13. The pulmonary drug delivery device of claim 12, wherein the medicine container is integrated into a medicine storage and delivery unit into which the droplet ejection device is also integrated.

14. A handheld pulmonary drug delivery device, comprising:
a drug delivery member including a drug delivery tube that defines a flow path, the drug delivery tube including a first tube in fluid communication with a second tube, the first tube and the second tube being arranged so as to form a sharp angle between each other;
a droplet ejection device configured to eject droplets of medication into the flow path from nozzles formed in an ejection head of the droplet ejection device;
a fan configured to generate airflow within the flow path, the airflow being configured to carry the ejected medication droplets along the flow path;
a pressure sensor configured to sense a pressure drop within the drug delivery tube indicative of user inhalation; and
a controller configured to control operation of the droplet ejection device and the fan relative to signals received from the pressure sensor.

15. The handheld pulmonary drug delivery device of claim 14, wherein the first and second tubes form an angle between each other of approximately 30 to 60 degrees.

16. The handheld pulmonary drug delivery device of claim 14, wherein the first and second tubes form an angle between each other of approximately 90 degrees.

17. The handheld pulmonary drug delivery device of claim 14, wherein a zone of relatively high turbulence exists adjacent an intersection of the first and second tubes due to the sharp angle formed between the first and second tubes.

18. The handheld pulmonary drug delivery device of claim 17, wherein the droplet ejection device is positioned so as to eject droplets of medication into the zone of relatively high turbulence.

19. The handheld pulmonary drug delivery device of claim 14, wherein the ejection head comprises heater resistors that cause the medication to be ejected from the nozzles.

20. The handheld pulmonary drug delivery device of claim 14, wherein the fan comprises a centrifugal blower.

21. The handheld pulmonary drug delivery device of claim 14, wherein the fan is mounted to the first tube and exhausts air directly into the first tube.

22. The handheld pulmonary drug delivery device of claim 14, further comprising an internal power supply that powers the droplet ejection device and the fan.

23. The handheld pulmonary drug delivery device of claim 14, further comprising a medicine container configured to supply medicine to the droplet ejection device.

24. The handheld pulmonary drug delivery device of claim 23, wherein the medicine container is provided on a medicine storage and delivery unit into which the droplet ejection device is integrated.

25. A handheld pulmonary drug delivery device, comprising:
an outer housing the defines an interior space;
a medicine storage and delivery unit provided within the interior space, the unit comprising an integrated container configured to hold medicine and an integrated droplet ejection device configured eject the medicine in fine droplets;
a drug delivery member provided within the interior space, the drug delivery member including a drug delivery tube that defines a flow path into which the medicine droplets can be injected, the drug delivery tube including a first tube and a second tube, the first tube and the second tube being arranged so as to form a sharp angle between each other that creates a zone of relatively high turbulence, the drug delivery member further comprising a support structure configured to support the medicine storage and delivery unit, the support structure including a platform to which the medicine storage and delivery unit mounts and a medicine injection tube along which the ejected droplets travel to the zone of relatively high turbulence;
a fan provided within the interior space, the fan being mounted to the first tube of the drug delivery tube and configured to generate airflow within the flow path, the airflow being configured to carry the ejected medication droplets along the flow path;
a pressure sensor provided within the interior space, the pressure sensor being configured to sense a pressure drop within the drug delivery tube indicative of user inhaling from the drug delivery tube; and
a controller provided within the interior space, the controller being configured to activate the droplet ejection device and the fan when user inhalation is detected such that medicine droplets injected into the airflow can be delivered with the airflow through the drug delivery tube and to the user's respiratory tract.

26. The handheld pulmonary drug delivery device of claim 25, wherein the first and second tubes form an angle between each other of approximately 30 to 60 degrees.

27. The handheld pulmonary drug delivery device of claim 25, wherein the first and second tubes form an angle between each other of approximately 90 degrees.

28. The handheld pulmonary drug delivery device of claim 25, wherein the droplet ejection device comprises heater resistors that cause the medication to be ejected from nozzles of the droplet ejection device.

29. The handheld pulmonary drug delivery device of claim 25, wherein the fan comprises a centrifugal blower.

30. The handheld pulmonary drug delivery device of claim 25, further comprising an power supply provided within the interior space that powers the droplet ejection device and the fan.

31. A method for administering a medication, comprising:
providing a drug delivery tube that comprises two tube sections that together define a flow path having a sharp bend;
forcing air into the drug delivery tube toward the sharp bend so as to generate a zone of relatively high turbulence adjacent the sharp bend;
injecting fine droplets of medication into the zone of relatively high turbulence to cause the droplets to shrink in size through evaporation; and
delivering the shrunken droplets along the flow path to the user.

32. The method of claim 31, wherein forcing air into the drug delivery tube comprises forcing the air with a fan.

33. The method of claim 32, wherein injecting fine droplets of medication comprises ejecting medication from a droplet ejection device.

34. The method of claim 33, wherein the drug delivery tube, fan, and droplet ejection device are each contained within a handheld pulmonary drug delivery device.
